(19) United States
(12) Patent Application Publication
(10) Pub. No.: US 2002/0025301 A1
(43) Pub. Date: Feb. 28, 2002

(54) Novel flavonoids and their use in cosmetic and dermatological preparations

(76) Inventors: Sylke Haremza, Neckargemund (DE); Frank Prechtl, Frankfurt (DE); Thorsten Habeck, Meckenheim (DE)

Correspondence Address: Herbert B. Keil, KEIL & WEINKAUF, 1101 Connecticut Ave., N.W., Washington, DC, 20036, US

(21) Appl. No.: 09/895,073

(22) Filed: Jul. 2, 2001

(30) Foreign Application Priority Data
Jul. 4, 2000 (DE) 10031809.6
Aug. 1, 2000 (DE) 10037846.3

(51) Int. Cl. A61K 7/42

(52) U.S. Cl. 424/59

(57) ABSTRACT

Flavonoids of the formula

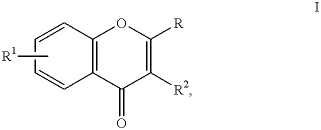

in which R is one of the radicals of the formula Ia or Ib

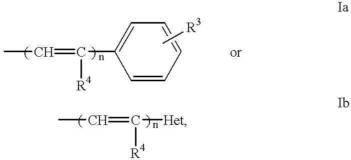

where

R¹ is hydrogen or C₁- to C₄-alkyl, hydroxyl, C₁- to C₄-alkoxy, phenyloxy or benzyloxy,

R² is hydrogen or C₁- to C₄-alkyl,

R³ is hydrogen, C₁- to C₄-alkyl, C₁- to C₄-alkoxy or cyano,

R⁴ is hydrogen, C₁- to C₄-alkyl or phenyl,

n is 0 or 1 and

Het is a pyridyl, furyl or methylpyrrolyl radical, with the proviso that when the radical R=Ia and n=0, at most 2 of the radicals R¹ to R³ are hydrogen at the same time,

and their use in cosmetic and dermatological preparations.

The invention relates to novel flavonoids, to processes for their preparation, to their use as UV-absorbers and as antioxidants in cosmetic and dermatological preparations and to these preparations.

A large number of flavonoids are already known from natural sources and via synthesis. The use in cosmetic and dermatological preparations has also already been described in JP 06 016 531.

However, the properties with regard to UV-absorption and antioxidant effect were still not entirely satisfactory. It is an object of the invention, therefore, to propose novel flavonoids which have improved application properties.

We have found that this object is achieved according to the invention with flavonoids of the formula I

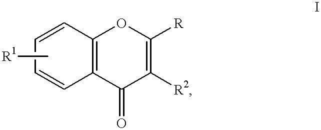

in which R is one of the radicals of the formula Ia or Ib

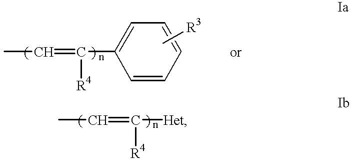

where

R¹ is hydrogen or C₁- to C₄-alkyl, hydroxyl, C₁- to C₄-alkoxy, phenyloxy or benzyloxy,

R² is hydrogen or C₁- to C₄-alkyl,

R³ is hydrogen, C₁- to C₄-alkyl, C₁- to C₄-alkoxy or cyano,

R⁴ is hydrogen, C₁- to C₄-alkyl or phenyl,

n is 0 or 1 and

Het is a pyridyl, furyl or methylpyrrolyl radical, with the proviso that when the radical R=Ia and n=0, at most 2 of the radicals R¹ to R³ are hydrogen at the same time.

Of the novel compounds, preference is given to those in which

n=1 and/or which have a radical R substituted by heterocycles.

The flavonoids of the formula I can be obtained by various synthesis routes, a common feature of which is, however, the starting material 2′-hydroxyacetophenone. In the following explanation of these routes, R¹, R², R³ and R⁴ are as defined above.

Method A: the Flavanone Route

In this synthesis route a 2′-hydroxyacetophenone derivative is reacted with a benzaldehyde derivative with catalysis from boric acid, silicon dioxide and piperidine to give a flavanone. The latter is then oxidized in a second reaction step using NBS/benzoyl peroxide to give the flavone.

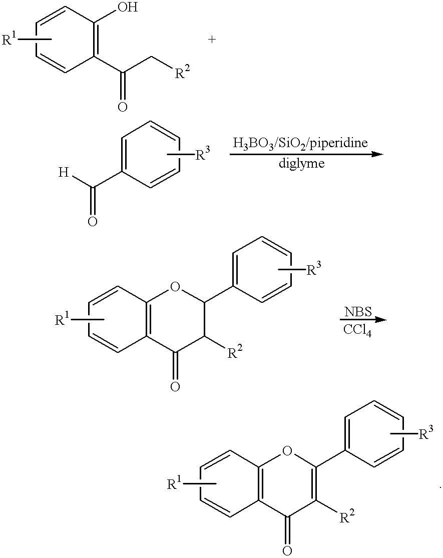

Method B: the Baker-Venkatamaran Route

Starting from a 2′-hydroxyacetophenone derivative, the reaction with an aromatic carbonyl chloride firstly gives the associated ester. The latter is then rearranged by a base-catalyzed Baker-Venkatamaran rearrangement to give a 1,3-dicarbonyl compound, which is finally cyclized with acid catalysis to give the flavone.

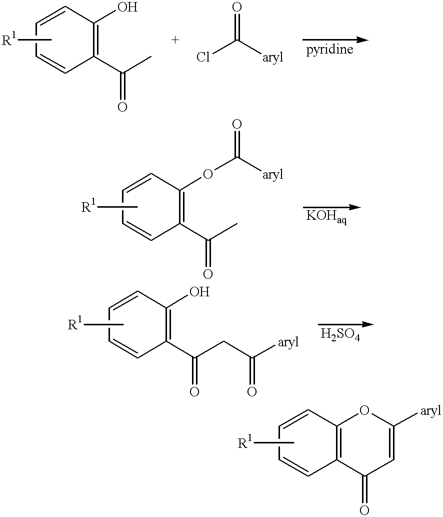

Method C: the DMSO Route

This synthesis route is used primarily for the synthesis of vinylogously lengthened flavonoids. For this, a cinnamaldehyde derivative is reacted with a 2′-hydroxyacetophenone derivative with base catalysis to give a vinylogously lengthened chalcone. The oxidative cyclization is finally carried out by heating in DMSO with the addition of catalytic amounts of iodine.

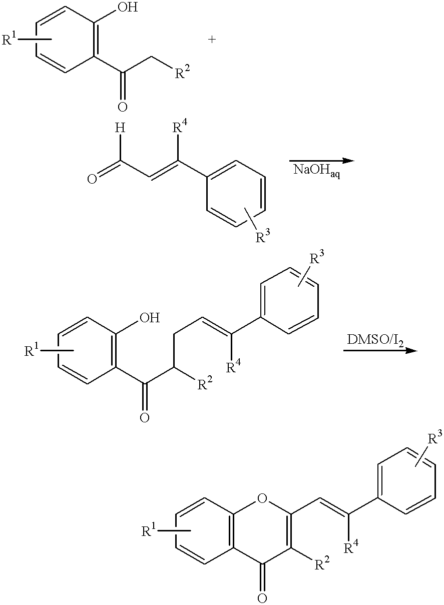

Method D: the Methylchromone Route

This synthesis route is used for the preparation of a number of vinylogously lengthened flavonoids. Here, the target compounds are prepared by base-catalyzed aldol condensation of a suitable aldehyde with a 2-methylchromone derivative.

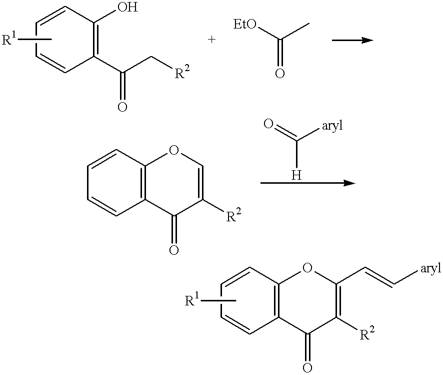

A reaction of ketones is not possible by this route.

Most of the synthesis routes described above can be carried out without a relatively large amount of synthetic preparatory work. However, for the synthesis of 5-hydroxy-3-methyl-substituted flavones, the time-consuming and labor-intensive preparation of 2′,6′-dihydroxypropiophenone is necessary. In the case of the preparation of flavonoids which carry hydroxyl substituents, additional reaction steps for the temporary protection (e.g. as methyl ether) and for the liberation of the hydroxyl groups may be necessary.

The flavonoids according to the invention can be systematically summarized using the structure types of the formulae II, III and IV, the substituents having the meanings given above.

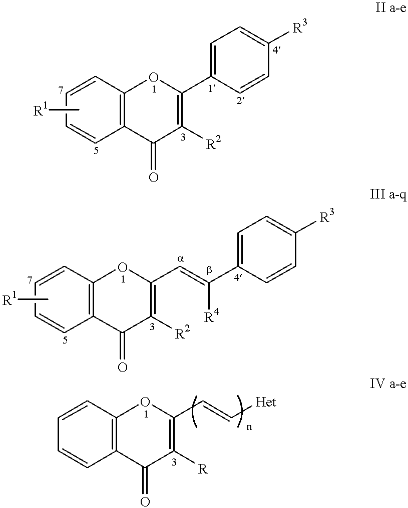

Tables 1 to 3 summarize the synthesis method and spectroscopic data of the flavonoids illustrated.

TABLE 1
Substitution pattern, synthesis route and spectroscopic data
(methanol) of the flavonoids of the formula II

| | | | | Synthesis | λₘₐₓ [nm] |
|---|---|---|---|---|---|
| No. | R² | R¹ | R³ | route | (1 g ε) |
| IIa | H | H | Methyl | B | 303 (4.44) |
| IIb | H | 6,8-Diethyl | Methoxy | B | 321 (4.42) |
| IIc | H | 5-Hydroxy | tert-Butyl | A | 336 (n.c.) |
| IId | Methyl | 5-Methoxy | H | A | 336 (n.c.) |
| IIe | Methyl | 5-Hydroxy | H | A | 336 (n.c.) |

TABLE 2
Substitution pattern, synthesis route and spectroscopic data
(methanol) of the vinylogous flavonoids of the formula III

| | | | | | Syn- | |
|---|---|---|---|---|---|---|
| | | | | | thesis | λₘₐₓ [nm] |
| No. | R² | R¹ | R³ | R⁴ | route | (1 g ε) |
| IIIa | H | H | H | H | D | 330 (4.60) |
| IIIb | Methyl | H | H | H | D | 338 (4.53) |
| IIIc | H | H | Cyano | H | D | 328 (4.48) |
| IIId | Methyl | H | Cyano | H | D | 338 (4.64) |
| IIIe | H | H | Methoxy | H | D | 360 (4.61) |
| IIIf | Methyl | H | Methoxy | H | D | 365 (4.57) |
| IIIg | H | H | Isopropyl | H | D | 345 (4.61) |
| IIIh | Methyl | H | Isopropyl | H | D | 349 (4.58) |
| IIIi | H | H | H | Phenyl | C | 336 (4.46) |
| IIIj | Methyl | H | H | Phenyl | C | 338 (4.35) |
| IIIk | H | 5-benzyl- | H | H | C | 330 (4.44) |
| | oxy | | | | | |
| IIIl | H | 5-Hydroxy | H | H | C | 334 (4.54) |
| IIIm | H | 5-Methoxy | H | Phenyl | C | 351 (4.14) |
| IIIn | H | 5-Hydroxy | H | Phenyl | C | 342 (4.32) |
| IIIo | Methyl | 5-Methoxy | H | Phenyl | C | 340 (4.31) |
| IIIp | Methyl | 5-Hydroxy | H | Phenyl | C | 343 (4.32) |
| IIIq | H | 6-Hydroxy | H | Phenyl | C | 339 (4.49) |

TABLE 3
substitution pattern, synthesis route and spectroscopic data
(methanol) of the flavonoids of the formula IV

| | R¹ | | | Synthesis | λₘₐₓ [nm] |
|---|---|---|---|---|---|
| No. | (R², R³, R⁴ = H) | n | Het | route | (1 g ε) |
| IVa | H | 1 | 2-furyl | D | 358 (4.53) |
| IVb | Methyl | 1 | 2-furyl | D | 364 (4.52) |
| IVc | H | 1 | 2-N- | D | 404 (n.c.) |
| | | | Methylpyrrolyl | | |
| IVd | Methyl | 1 | 4-Pyridyl | D | 323 (n.c.) |
| IVe | H | 0 | 3-Pyridyl | B | 358 (4.53) |

The present application also provides for the use of the flavonoids of the formula I in cosmetic or dermatological preparations, and these preparations as substance mixtures which comprise effective amounts of the flavonoids of the formula I, i.e., for example, 0.1 to 10% by weight, preferably 0.5 to 10% by weight and in particular 1 to 7% by weight, based on the total amount of the cosmetic and dermatological preparation.

The cosmetic and dermatological preparations are usually based on a carrier which comprises at least one oil phase. However, preparations solely on an aqueous basis are also possible in the case of the use of compounds containing hydrophilic substituents. Accordingly, oils, oil-in-water and water-in oil emulsions, creams and pastes, lip protection stick compositions or grease-free gels are suitable.

Such preparations can accordingly be in liquid, paste or solid form, for example as water-in-oil creams, oil-in-water creams and lotions, aerosol foam creams, gels, oils, grease sticks, powders, sprays or alcoholic-aqueous lotions.

Customary oil components in cosmetics are, for example, paraffin oil, glyceryl stearate, isopropyl myristate, diisopropyl adipate, cetyl stearyl 2-ethylhexanoate, hydrogenated polyisobutene, vaseline, caprylic/capric triglycerides, microcrystalline wax, lanolin and stearic acid.

Customary cosmetic auxiliaries which are suitable as additives are, for example, coemulsifiers, fats and waxes, stabilizers, thickeners, biogenic active ingredients, film formers, fragrances, dyes, pearlizing agents, preservatives, pigments, electrolytes (e.g. magnesium sulfate) and pH regulators. Suitable coemulsifiers are, preferably, known W/O and also O/W emulsifiers, such as, for example, polyglycerol esters, sorbitan esters or partially esterified glycerides. Typical examples of fats are glycerides; suitable waxes include beeswax, paraffin wax and microcrystalline waxes, optionally in combination with hydrophilic waxes. Stabilizers which may be used are metal salts of fatty acids, such as, for example, magnesium stearate, aluminum stearate and/or zinc stearate. Suitable thickeners are, for example, crosslinked polyacrylic acids and derivatives thereof, polysaccharides, in particular xanthan gum, guar guar, agar agar, alginates and tyloses, carboxymethylcellulose and hydroxyethylcellulose, and also fatty alcohols, monoglycerides and fatty acids, polyacrylates, polyvinyl alcohol and polyvinylpyrrolidone. Biogenic active ingredients is understood as meaning, for example, plant extracts, protein hydrolyzates and vitamin complexes. Customary film formers are, for example, hydrocolloids, such as chitosan, microcrystalline chitosan or quaternary chitosan, polyvinylpyrrolidone, vinylpyrrolidone/vinyl acetate copolymers, polymers of the acrylic acid series, quaternary cellulose derivatives and similar compounds. Examples of suitable preservatives are formaldehyde solution, p-hydroxy benzoate or sorbic acid. Examples of suitable pearlizing agents are glycol distearic esters, such as ethylene glycol distearate, but also fatty acids and fatty acid monoglycol esters. Dyes which may be used are the substances approved and suitable for cosmetic purposes, as listed, for example, in the publication “Kosmetische Farbemittel” [Cosmetic Colorants] from the Farbstoffkommission der Deutschen Forschungsgemeinschaft [Dyes Commission of the German Research Society], published by Verlag Chemie, Weinheim, 1984. These dyes are usually used in concentrations of from 0.001 to 0.1% by weight, based on the total mixture.

The total proportion of auxiliaries and additives can be 1 to 80% by weight, preferably 6 to 40% by weight and the nonaqueous proportion (“active substance”) can be 20 to 80% by weight, preferably 30 to 70% by weight, based on the composition. The compositions can be prepared in a manner known per se, i.e. for example by hot, cold, hot-hot/cold-cold or PIT emulsification. This is a purely mechanical process, and no chemical reaction takes place.

Finally, it is possible to co-use further substances known per se which absorb in the UV region, provided they are stable in the overall system of the combination of UV filters that is to be used according to the invention.

The majority of the light protection agents in the cosmetic and dermatological preparations is to protect the human epidermis consists of compounds which absorb UV light in the UV-B region, i.e. in the range from 280 to 320 nm. For example, the proportion of the UV-A absorber to be used according to the invention is 10 to 90% by weight, preferably 20 to 50% by weight, based on the total amount of UV-B and UV-A absorbing substances.

Suitable UV filter substances which are used in combination with the compounds of the formula I to be used according to the invention are any UV-A and UV-B filter substances. Examples are:

| | | CAS No. |
|---|---|---|
| No. | Substance | (= acid) |
| 1 | 4-Aminobenzoic acid | 150-13-0 |
| 2 | 3-(4′-Trimethylammonium)benzylidenebornan- | 52793-97-2 |
| | 2-one methylsulfate | |
| 3 | 3,3,5-Trimethylcyclohexyl salicylate | 118-56-9 |
| | (homosalate) | |
| 4 | 2-Hydroxy-4-methoxybenzophenone | 131-57-7 |
| | (oxybenzone) | |
| 5 | 2-phenylbenzimidazole-5-sulfonic acid and its | 27503-81-7 |
| | potassium, sodium and triethanolamine salts | |
| 6 | 3,3′-(1,4-Phenylenedimethine)bis(7,7-dimethyl- | |
| | 2-oxobicyclo[2.2.1]heptane-1-methanesulfonic | 90457-82-2 |
| | acid) and its salts | |
| 7 | Polyethoxyethyl 4-bis (polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethyihexyl 4-methoxycinnamate | 5466-77-3 |
| 12 | 2-Hydroxy-4-methoxybenzophenone-5-sulfonic acid | 4065-45-6 |
| | (sulisobenzone) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane- | 63260-25-9 |
| | 1,3-dione | |
| 16 | 4-Isopropylbenzyl salicylate | 94134-93-7 |
| 17 | 2,4,6-Trianiline (o-carbo-2′-ethylhexyl- | 88122-99-0 |
| | 1′-oxy)-1,3,5-triazine | |
| 18 | 3-Imidazol-4-ylacrylic acid and its ethyl ester | 104-98-3 |
| 19 | Ethyl 2-cyano-3,3-diphenylacrylate | 5232-99-5 |
| 20 | 2-Ethylhexyl 2-cyano-3, 3-diphenylacrylate | 6197-30-4 |
| 21 | Menthyl-o-aminobenzoate or: | 134-09-8 |
| | 2,5-methyl-2-(1-methylethyl)-2-aminobenzoate | |
| 22 | Glyceryl p-aminobenzoate or: | 136-44-7 |
| | 1-glyceryl 4-aminobenzoate | |
| 23 | 2,2′-Dihydroxy-4-methoxybenzophenone- | 131-53-3 |
| | (dioxybenzone) | |
| 24 | 2-Hydroxy-4-methoxy-4-methylbenzophenone- | 1641-17-4 |
| | (mexenone) | |
| 25 | Triethanolamine salicylate | 2174-16-5 |
| 26 | Dimethoxyphenylglyoxalic acid or: | 4732-70-1 |
| | sodium 3,4-dimethoxyphenyiglyoxalate | |
| 27 | 3-(4′-sulfobenzylidene)bornan-2-one and its | 56039-58-8 |
| | salts | |
| 28 | 4-tert-Butyl-4′-methoxy-dibenzoylmethane | 70356-09-1 |
| 29 | 2,2′,4,4′-tetrahydroxybenzophenone | 131-55-5 |
| 30 | 2,2′-methylene-bis[6(2H-bezotriazol-2-yl)-4- | 103597-45-1 |
| | (1,1,3,3,-tetramethylbutyl)phenol] | |
| 31 | 2,2′-(1,4-phenylene)-bis-1H-benzimidazole-4,6- | 180898-37-7 |
| | disulfonic acid, sodium salt | |
| 32 | 2,4-bis[4-(2-Ethylhexyloxy)-2-hydroxylphenyl- | 187393-00-6 |
| | 6-(4-methoxyphenyl)-(1,3,5)-triazine | |
| 33 | 3-(4-Methylbenzylidene)camphor | 36861-47-9 |
| 34 | Polyethoxyethyl 4-bis(polyethoxy)paraamino- | 113010-52-9 |
| | benzoate | |
| 35 | 2,4-Dihydroxybenzophenone | 131-56-6 |
| 36 | 2,2′-Dihydroxy-4,4′-dimethoxybenzophenone-5,5′- | 3121-60-6 |
| | disodium sulfonate | |

Finally, micronized pigments, such as titanium dioxide and zinc oxide, are also to be mentioned.

To protect human hair against UV rays, the light protection agents of the formula I according to the invention can be incorporated into shampoos, lotions, gels, hairsprays, aerosol foam creams or emulsions in concentrations of from 0.1 to 10% by weight, preferably 1 to 7% by weight. The respective formulations can be used here inter alia for the washing, coloring and for the styling of hair.

The compounds to be used according to the invention are generally notable for a particularly high absorption capacity both in the UV-A and UV-B radiation range. Furthermore, they are readily soluble in cosmetic oils and can be readily incorporated into cosmetic formulations. The emulsions prepared with the compounds I are notable in particular for their high stability, the compounds I themselves by their high photostability, and the preparations prepared with I by their pleasant feel on the skin.

The UV filter action of the compounds of the formula I according to the invention can also be utilized for stabilizing active ingredients and auxiliaries in cosmetic and dermatological formulations.

In addition, the flavonoids of the formula I according to the invention have antioxidative properties and can at least partially replace or enhance or improve the action of the antioxidants customarily used.

Antioxidants which are customarily used are, for example, chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg-ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, butylhydroxytoluene, butylhydroxyanisol, nordihydroguaiacic acid, nordihydroguaiaretic acid, trihydroxybutyrophenone, uric acid and derivatives thereof, mannose and derivatives thereof, sesamol, sesamolin, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention. The amount of the abovementioned antioxidants in the preparations, alone or in combination is, for example, 0.001 to 30% by weight, preferably 0.1 to 20% by weight and in particular 0.1 to 5% by weight.

### EXAMPLES

Preparation of the Flavonoids

### Example 1

(Method A)

Preparation of 4′-tert-butyl-5-hydroxyflavone

Stage 1: 4′-tert-butyl-5-hydroxyflavanone

25.0 g (420 mmol) of silicon dioxide, 5.9 g (80 mmol) of boric acid and 1.1 g (13 mmol) of piperidine are initially introduced under a nitrogen atmosphere. To this are added 7.7 g (50 mmol) of 2,6-dihydroxyacetophenone and 8.1 g (50 mmol) of 4-tert-butylbenzaldehyde in 100 ml of diethylene glycol dimethyl ether. The reaction mixture is then heated at 120° C. for 12 hours. The mixture is then left to cool and diluted with 100 ml of acetone. The silicon dioxide is filtered off and washed with 3×50 ml of acetone. The combined filtrates are concentrated by evaporation, and the solid which remains is taken up in 50 ml of ethanol. To isolate the product, the mixture is poured onto 750 ml of water, and the pH is rendered acidic using hydrochloric acid. The yellow solid which precipitates out is filtered off with suction and washed with a small amount of methanol. Recrystallization is carried out from methanol.

Yield: 11.1 g (40.0 mmol, 74%) of yellow powder

m.p.: 88° C.

Stage 2: 5-acetoxy-4′-tert-butylflavanone

2.8 g (9.5 mmol) of 4′-tert-butyl-5-hydroxyflavanone are heated with 4.9 g (48 mmol) of acetic anhydride for 2 hours in 5 ml of pyridine with reflux condensation. The hot solution is then poured onto ice, and the precipitate which forms is filtered off and recrystallized from methanol.

Yield: 3.1 g (9.2 mmol, 97%)

m.p.: 180° C.

Stage 3: 4′-tert-butyl-5-hydroxyflavone

In 200 ml of tetrachloromethane, 3.8 g (10 mmol) of 5-acetoxy-4′-tert-butylflavanone are heated with 2.7 g (15 mmol) of NBS and a spatula tip of benzoyl peroxide for 2 hours with reflux condensation. The mixture is then cooled in an ice bath, and the succinimide which forms is filtered off. The filtrate is concentrated by evaporation, and 200 ml of a 5% strength methanolic sodium hydroxide solution are added. The mixture is then heated for 30 minutes with reflux condensation, before the solvent is again stripped off. The residue is taken up in 250 ml of water, and the pH is rendered acidic using 2N hydrochloric acid. The product is extracted with ethyl acetate and, after drying over magnesium sulfate and stripping the solvent off, is recrystallized from acetone.

Yield: 1.25 g (4.3 mmol, 43%)

m.p.: 146° C.

UV-vis (methanol): λmax (lg ε): 336 nm (n.c.)

### Example 2

(Method B)

Preparation of 4′-methylflavone

Stage 1: 2-acetylphenyl 4-methylbenzoate

In a round flask, 13.6 g (100 mmol) of 2-hydroxyacetophenone are dissolved with 21.1 g (136 mmol) of 4-methylbenzoyl chloride in 20 ml of absolute pyridine, and stirred with the exclusion of moisture. The temperature of the reaction mixture increases rapidly, and a colorless solid precipitates out, which turns violet as the reaction proceeds. After 20 min., the reaction mixture is poured, with vigorous stirring, onto 600 ml of 3% strength hydrochloric acid and 200 g of ice. The pale violet colored precipitate which forms is filtered off, washed firstly with 20 ml of methanol, then with 20 ml of water and recrystallized from methanol.

Yield: 21.6 g (85 mmol, 85%) of colorless crystals

m.p.: 98° C.

Stage 2: 1-(2-hydroxyphenyl)-3-p-tolylpropane-1,3-dione

To a solution, heated to 50° C., of 20.0 g (83 mmol) of 2-acetylphenyl 4-methylbenzoate in 75 ml of absolute pyridine are added, with stirring, 7.0 g (125 mmol) of powdered potassium hydroxide. After 15 min., the reaction mixture is cooled to room temperature, and the mixture is acidified with 100 ml of 10% strength acetic acid. The yellow solid which forms is filtered off and dried. It is reacted further without further purification operations.

Yield: 16.0 g (63 mmol, 80%) of yellow powder

m.p.: 108° C.

Stage 3: 4′-methylflavone

To a solution of 16.0 g (63 mmol) of 1-(2-hydroxyphenyl)-3-p-tolylpropane-1,3-dione in 90 ml of glacial acetic acid are added, with stirring, 3.5 ml of concentrated sulfuric acid. The mixture is heated at 100° C. for 1 hour, and the reaction mixture is then poured, with vigorous stirring, onto 500 g of ice. The gray solid which forms is filtered off with suction, washed with copious amounts of water and recrystallized from methanol.

Yield: 13.5 g (57 mmol, 91%) of colorless powder

m.p.: 117° C.

UV-vis (methanol): λmax (lg ε): 303 nm (4.44)

### Example 3

(Method C)

Preparation of 5-hydroxy-2-styrylchromone

Stage 1: 2′-benzyloxy-6′-hydroxy-2-cinnamylideneacetophenone

To a solution of 1.5 g (6.2 mmol) of 2′-benzyloxy-6′-hydroxyacetophenone in 25 ml of methanol are slowly added, with stirring, 25 ml of an aqueous 60% strength sodium hydroxide solution. Then, at room temperature 1.0 g (7.5 mmol) of cinnamaldehyde in 10 ml of methanol are added over the course of 15 min., and the mixture is stirred for 20 hours at room temperature. The reaction mixture is then poured onto 150 g of ice, and the pH is adjusted to 2 with 2N hydrochloric acid. The solid which precipitates out is filtered off. It is dissolved in 200 ml of chloroform and washed with 2×200 ml of a 5% strength aqueous sodium hydrogen carbonate solution. After drying over magnesium sulfate, the chloroform of stripped off, and the solid which remains is recrystallized from ethanol.

Yield: 0.9 g (2.5 mmol, 40%) of orange-colored needles

m.p.: 135° C.

Stage 2: 5-benzyloxy-2-styrylchromone

To a solution of 720 mg (2.02 mmol) of 2′-benzyloxy-6′-hydroxy-2-cinnamylideneacetophenone in 10 ml of DMSO are added 20 mg (79 μmol) of iodine, and the mixture is refluxed for 2 hours. It is then poured onto 300 ml of iced water, and the solid which precipitates out is filtered off. It is then taken up in 150 ml of chloroform and washed twice with 20% strength aqueous sodium thiosulfate solution and once with water. After drying over magnesium sulfate and stripping the solvent off, the product is recrystallized from ethanol.

Yield: 310 mg (0.87 mmol, 44%) of colorless needles

m.p.: 178-182° C.

Stage 3: 5-hydroxy-2-styrylchromone

To an ice-cooled solution of 330 mg (0.93 mmol) of 5-benzyloxy-2-styrylchromone in 10 ml of dichloromethane are added dropwise, with stirring, 500 mg (2.00 mmol) of boron tribromide in 5 ml of dichloromethane. The mixture is then heated slowly to room temperature and stirred at room temperature until the reaction is complete (TLC control, 3 days). For the isolation, the reaction mixture is poured into 25 ml of ice water, and the organic phase is separated off, washed with water and dried over magnesium sulfate. The solid which remains after the solvent has been stripped off is recrystallized from ethanol.

Yield: 130 mg (0.49 mmol, 53%) of yellow crystals

m.p.: 155° C.

UV-vis (methanol): λmax (lg ε): 334 nm (4.54)

### Example 4

(Method D)

Preparation of 2-(4′-methoxycinnamyl)chromone

400 mg (2.50 mmol) of 2-methylchromone are added to 25 ml of a 0.2 molar ethanolic sodium ethoxide solution, and the mixture is stirred for 15 minutes at room temperature. 410 mg (3.01 mmol) of 4-methoxybenzaldehyde are then added, and the reaction mixture is stirred for 24 hours at room temperature. During this time, a yellow solid precipitates out. To isolate the product, the reaction mixture is poured into 25 ml of ice water, and the product is filtered off. Recrystallization is carried out from ethanol.

Yield: 590 mg (2.12 mmol, 85%) of yellow powder

m.p.: 136° C.

UV-vis (methanol): λmax (lg ε): 360 nm (4.61)

The other compounds in Tables 1 to 3 are obtained in a similar manner.

### Application Examples

Example 5 - Soft Skin Fluid

| | % w/w |
|---|---|
| Ceteareth-6 and Stearyl Alcohol | 2.50 |
| Ceteareth-25 | 2.50 |
| Hydrogenated Coco-Glycerides | 1.50 |
| PEG-40 Dodecyl Glycol Copolymer | 3.00 |
| Dimethicone | 3.00 |
| Phenethyl Dimethicone | 2.00 |
| Cyclomethicone | 2.00 |
| Cetearyl Octanoate | 5.00 |
| Avocado Oil | 1.00 |
| Sweet Almond Oil | 2.00 |
| Wheat Germ Oil | 0.80 |
| Panthenol USP | 1.00 |
| Phytantriol | 0.20 |
| Tocopheryl Acetate | 0.30 |
| Propylene Glycol | 5.00 |
| Perfume | q.s. |
| Preservative | q.s. |
| Flavonoid of the formula IIIb | 0.20 |
| Aqua | 69.20 |

Example 6 - Hand Protection Cream

| | % w/w |
|---|---|
| Cetearyl Alcohol | 1.00 |
| Glyceryl Stearate | 1.50 |
| Stearyl Alcohol | 1.50 |
| Cetyl Palmitate | 2.00 |
| Tocopheryl Acetate | 0.50 |
| Dimethicone | 8.00 |
| Ceteareth-6 and Stearyl Alcohol | 3.00 |
| Octyl Methoxycinnamate | 5.00 |
| Propylene Glycol | 8.00 |
| Panthenol | 1.00 |
| Evening Primrose Oil | 3.00 |
| PEG-7 hydrogenated Castor Oil | 6.00 |
| Glyceryl Oleate | 1.00 |
| Phenethyl Dimethicone | 3.00 |
| Beeswax | 1.50 |
| Locust Bean Gum | 0.80 |
| Silk powder | 0.80 |
| Preservative | q.s. |
| Perfume | q.s. |
| Borax | 0.10 |
| Flavonoid of the formula IIId | 2.00 |
| Aqua | 52.30 |

Example 7 - Sun Care Lotion

| | % w/w |
|---|---|
| PEG-7 hydrogenated Castor Oil | 6.00 |
| PEG-40 hydrogenated Castor Oil | 0.50 |
| Isopropyl Palmitate | 7.00 |
| PEG-45/Dodecyl Glycol Copolymer | 2.00 |
| Jojoba Oil | 3.00 |
| Magnesium Stearate | 0.60 |
| Octyl Methoxycinnaniate | 8.00 |
| C12-15 Alkyl Benzoate | 5.00 |
| Titanium Dioxide | 4.00 |
| Propylene Glycol | 5.00 |
| EDTA | 0.20 |
| Preservative | q.s. |
| Aqua | 57.20 |
| Sodium Ascorbyl Phosphate | 1.00 |
| Tocopheryl Acetate | 0.50 |
| Flavonoid of the formula IIc | 5.00 |
| Perfume | q.s. |

We claim:

1. A flavonoid of the formula I 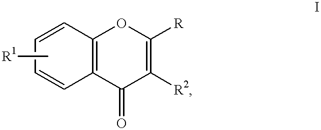
in which R is one of the radicals of the formula Ia or Ib 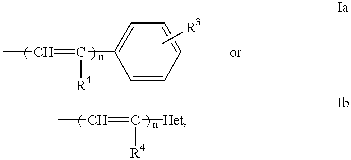
where
R¹ is hydrogen or C₁- to C₄-alkyl, hydroxyl, C₁- to C₄-alkoxy, phenyloxy or benzyloxy,
R² is hydrogen or C₁- to C₄-alkyl,
R³ is hydrogen, C₁- to C₄-alkyl, C₁- to C₄-alkoxy or cyano,
R⁴ is hydrogen, C₁- to C₄-alkyl or phenyl,
n is 0 or 1 and
Het is a pyridyl, furyl or methylpyrrolyl radical, with the proviso that when the radical R=Ia and n=0, at most 2 of the radicals R¹ to R³ are hydrogen at the same time.

2. The use of flavonoids of the formula I as claimed in claim 1 as UV-absorbers and/or antioxidants in cosmetic or dermatological preparations.

3. A cosmetic or dermatological preparation comprising effective amounts of at least one flavonoid of the formula I 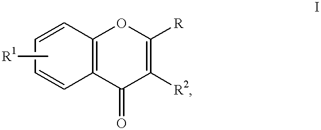
in which R is one of the radicals of the formula Ia or Ib 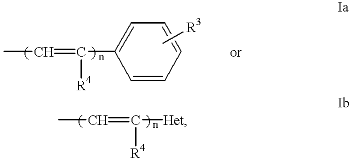
where
R¹ is hydrogen or C₁- to C₄-alkyl, hydroxyl, C₁- to C₄-alkoxy, phenyloxy or benzyloxy,
R² is hydrogen or C₁- to C₄-alkyl,
R³ is hydrogen, C₁- to C₄-alkyl, C₁- to C₄-alkoxy or cyano,
R⁴ is hydrogen, C₁- to C₄-alkyl or phenyl,
n is 0 or 1 and
Het is a pyridyl, furyl or pyrrolyl radical, with the proviso that when the radical R=Ia and n=0, at most 2 of the radicals R¹ to R³ are hydrogen at the same time.

4. A cosmetic or dermatological preparation as claimed in claim 3, having a content of at least 0.1% by weight, based on the finished preparation, of a flavonoid of the formula I as claimed in claim 1.
